(19) United States
(12) Patent Application Publication
(10) Pub. No.: US 2024/0216599 A1
(43) Pub. Date: Jul. 4, 2024

(54) EXTRACORPOREAL CIRCUIT SUPPORT

(71) Applicants: Michael HOFMANN, Zumikon (CH); Martin Oliver SCHMIADY, Thalwil (CH); Michael HÜBLER, Hornbrechtikon (CH); EIDGENÖSSISCHE TECHNISCHE HOCHSCHULE ZÜRICH, Zürich (CH)

(72) Inventors: Michael Hofmann, Zumikon (CH); Samuel SOLLBERGER, Zürich (CH); Martin Oliver SCHMIADY, Thalwil (CH); Marianne SCHMID, Schaffhausen (CH); Mirko MEBOLDT, Konstanz (DE)

(73) Assignees: Michael HOFMANN, Zumikon (CH); Martin Oliver SCHMIADY, Thalwil (CH); Michael HÜBLER, Hornbrechtikon (CH); ETH-Eidgenössische Technische Hochschule Zürich, Zürich (CH)

(21) Appl. No.: 18/570,845

(22) PCT Filed: Oct. 29, 2022
(86) PCT No.: PCT/EP22/67958

§ 371 Date: Dec. 15, 2023

(30) Foreign Application Priority Data
Jun. 29, 2021 (DE) 10 2021 116 747.7

(51) Int. Cl. A61M 1/36

(52) U.S. Cl. CPC A61M 1/3644

(57) ABSTRACT

An extracorporeal circuit support with a main liquid pump, the inlet of which can be connected to the blood circuit of a patient via at least one first liquid line and the outlet of which can be connected to the blood circuit via at least one second liquid line, an oxygenator for enriching the blood being conducted in the at least one second liquid line with oxygen, and a pump drive which drives the main liquid pump. The extracorporeal circuit support has, in addition to the main blood pump or main liquid pump and the oxygenator, the pump drive designed to be MR-conditional, i.e., MR-compliant under specific conditions, and is designed in the form of a gas expansion motor. The extracorporeal circuit support can be used as a heart-lung machine or as an ECMO device

### CROSS-REFERENCE TO RELATED APPLICATIONS

This application is a 371 National Phase of International application No. PCT/EP2022/067958, filed Jun. 29, 2022, which claims priority to German Patent Application No. 10 2021 116 747.7, filed Jun. 29, 2021, both of which are incorporated herein by reference as if fully set forth.

### TECHNICAL FIELD

The invention relates to an extracorporeal mechanical circuit support with a cardiovascular system and lung function, having a liquid primary pump, the pump inlet of which can be connected via at least one first liquid line and the pump outlet of which can be connected via at least one second liquid line to the circulatory system of a patient, having an oxygenator for enriching with oxygen the blood conveyed in the at least one second liquid line to the patient, and having a pump drive which drives the liquid primary pump.

The extracorporeal mechanical circuit support may also be referred to as apparatus for extracorporeal mechanical circuit support.

The invention furthermore relates to an MRT arrangement having a magnetic resonance tomograph (MRT) and an extracorporeal circuit support.

### BACKGROUND

Technically, the extracorporeal heart-lung circuit is often implemented with heart-lung machines. These are needed in order to replace the pump function of the heart and the lung functions in respect of oxygen enrichment (oxygenation) of the blood and in respect of carbon dioxide elimination for a limited period of time so as, for example, to allow an open-heart surgery operation. In this case, the blood is delivered via a cardiopulmonary bypass to the extracorporeal heart-lung machine, enriched with oxygen and rid of carbon dioxide in the latter, and subsequently delivered back to the body of the patient in a circuit. This is because the heart cannot perform its basic function of blood supply during the period of time of the open-heart surgery operation, the heart-lung machine is used so that all organs can nevertheless be supplied with blood and therefore with oxygen. This heart-lung machine consists mainly of a liquid primary pump also serving as an artificial heart (=blood pump) and an artificial lung (=oxygenator), in which case a flow sensor may measure the blood flow and a pressure sensor may measure the blood pressure.

At present, essentially two types of pump are used as liquid primary pumps or blood pumps, namely
rotary pumps, also known as centrifugal pumps or axial-flow pumps, and
peristaltic pumps, also known as roller pumps or flexible-tube pumps.

Heart-lung machines are essentially used only during such open-heart surgery operations. For patients who in the short or medium term have a weak heart or a weak lung, ECMO devices (ECMO=extracorporeal membrane oxygenator) are used as support for the weak organs in the sense of an oxygenator arranged outside the body of the patient. Like the heart-lung machine, an ECMO circuit also consists of a blood pump, oxygenator and heat exchanger, but is more compact, simpler and also portable.

Such operations and other emergency and intensive care interventions are intended in the future to be carried out in conjunction with a magnetic resonance tomography of the patient and to deliver the information relating to the extent and localization of ischemic regions, the vascular state and the differentiation of a possible recovery of the tissue at risk. In this case, a conditionally MR-compatible cardiopulmonary support may be necessary, which ensures safety and functions properly alongside a magnetic resonance tomograph (MRT). Although examinations can already be carried out nowadays with heart-lung machines in an MRT, significant modifications and safety precautions are required therefor with relocation of the entire system—except for the tube system. However, the blood pump per se is the essential component of the heart-lung machine and it should always be capable of being operated close to the patient. An MR-compatible blood pump is not currently available.

So that a blood pump can be operated in the vicinity of the MRT, the following criteria must be satisfied:
no or scarcely any ferromagnetic components, since these are attracted by the MRT; attracted components may easily lead to projectiles and therefore constitute a safety risk.
no or scarcely any electrically conductive components which are moved or rotated, since otherwise eddy currents which may torque or heat the components occur.
no electromagnetic drive.

A device with the classification MR conditional, that is to say a conditionally MR-compatible or conditionally MR-suitable device, must according to the standard ASTM F2503 generate no artifacts during the imaging, and is located outside the image region.

Since conventional extracorporeal circuits are to date not/conditionally MR-compatible or MR-safe, and since the roller pumps and axial-flow pumps used as the primary liquid pump in the conventional extracorporeal circuits have all not been MR-compatible, until now the cardiopulmonary bypass for use in the MRT had been implemented by means of very long blood tubes or with a pump head which has been driven via a long fiber-reinforced composite shaft by an electric motor installed away from the MRT. Such solutions are incompatible with clinical use.

### SUMMARY

It is therefore an object in particular to provide an extracorporeal circuit support of the type mentioned in the introduction which may be compactly designed and used for MR-conditional cardiopulmonary support even with small patients, for example neonates and infants.

The achievement according to the invention of this object consists in particular in that the pump drive is designed at least conditionally MR-compatibly and is configured as a gas expansion motor. This also constitutes an MR-safe design.

The extracorporeal circuit support according to the invention also has a blood or liquid primary pump, the pump inlet of which can be connected via at least one first liquid line and the pump outlet of which can be connected via at least one second liquid line to the circulatory system of a patient. In this case, the blood or liquid primary pump of the extracorporeal circuit support according to the invention is assigned a pump drive which drives the liquid primary pump. According to the invention, this pump drive is also designed conditionally MR-compatibly and is therefore configured as a gas expansion motor. Such a gas expansion motor may also be constructed without interfering ferromagnetic components which would otherwise be attracted by the MRT. Furthermore, the gas expansion motor used as a pump drive makes do without interfering electrically conductive components which are moved or rotated, and which otherwise could generate eddy currents. In particular, the gas expansion motor used as a pump drive has no electromagnetic drive so that in the extracorporeal circuit support according to the invention, besides the liquid primary pump and the oxygenator, the pump drive is also designed conditionally MR-compatibly and may be used in the environment of an MRT device. In this case, the extracorporeal circuit support according to the invention also has an oxygenator for enriching with oxygen the blood conveyed in the at least one second liquid line that leads to the patient. The extracorporeal circuit support according to the invention may be used as heart-lung machine or as an ECMO device.

In order to be able to control and regulate the volume flow rate of the compressed gas supplied to the gas expansion motor as the working medium, it is advantageous that this volume flow rate can be regulated by means of at least one proportional valve, in particular a piezo valve.

In order to convert the rotational movement of the pump drive, which is used as a driving force, into a pump movement of the blood pump, it is advantageous for the gas expansion motor to have a drive shaft which is operatively connected to the liquid primary pump.

In order to ensure the circuit support of the patient in the event of a failure of the power supply of the pump drive, it is advantageous for at least one connector for an emergency crank to be provided on the drive train. Likewise, a preferably removable emergency crank may be provided. By means of the emergency crank, the liquid primary pump can be driven manually. This may, for example, be necessary when, during mobile operation of the circuit support by using a gas cartridge for driving the gas expansion motor, the gas cartridge is emptied.

Since the gas expansion motor cannot generate arbitrarily high and low rotational speeds and torques, according to one preferred embodiment according to the invention a transmission is provided in the drive train between the gas expansion motor and the liquid primary pump. This transmission transmits the torque generated by the drive to the pump head.

It may in this case be advantageous for the transmission to be designed as a continuously variable transmission, as a gear transmission or planetary transmission, or as a hydrodynamic torque converter.

It may be advantageous for the aforementioned connector for the emergency crank and/or the emergency crank in this case to be arranged in the drive train before a transmission, for example before the aforementioned transmission, which may be interconnected between the gas expansion motor and the liquid primary pump. Thus, a drive movement applied manually via the emergency crank may be applied via the transmission and converted by the latter. By this transmission support, it may be more readily possible to achieve a particular minimum rotational speed of the liquid primary pump, which may be necessary for proper operation of the liquid primary pump, during manual emergency operation as well.

In order as far as possible to avoid further interfering effects of the extracorporeal circuit support according to the invention used in the environment of an MRT, it is advantageous for the drive shaft and preferably all shafts provided in the drive train to be produced from preferably stiff fiber-reinforced composites.

For the same purpose, it may be expedient for the drive shaft and preferably all shafts provided in the drive train to be mounted in ceramic ball bearings and/or in plastic bushes.

It may be advantageous for the liquid primary pump, which propels the medium, to be configured as a roller pump, peristaltic pump or flexible-tube pump. The medium is in this case contained in a tube, which is used as a liquid line, of the cardiopulmonary bypass, one or more rollers of this liquid primary pump rolling over the tube and thereby propelling the medium contained in the tube.

Occlusive and non-occlusive roller pumps may be used, the tube used being entirely compressed in the case of occlusive roller pumps, while non-occlusive roller pumps press the tube either not completely or at least not against an outer contour.

According to a further advantageous embodiment according to the invention, the liquid primary pump is configured as a dynamic pump. In this case, this dynamic pump may be configured as a rotary pump or as an axial-flow pump. It is also possible for the liquid primary pump to be configured as an impeller pump. For use of the machine according to the invention also as an ECMO machine, balloon pulsation may additionally be used.

In order to ensure safe use of the extracorporeal circuit support in conjunction with an MRT examination, it is advantageous for moving parts of the transmission and/or of the liquid primary pump to be designed to be metal-free. The use of ferromagnetic substances may thus be avoided. The moving parts may, for example, consist of plastic and/or ceramic. It is particularly advantageous for the moving parts to be configured to be MR-safe. A circuit support in which drive-relevant metal parts are installed neither in the transmission nor in the liquid primary pump may thus be produced.

It is advantageous for compressed air or nitrogen to be usable as the working medium for the gas expansion motor. Since compressed air is available in any operating room and any MRT room, the use of compressed air is also envisioned as the working medium for the gas expansion motor of the extracorporeal circuit support according to the invention. It would also be conceivable to use nitrogen instead of compressed air as the compressed gas.

In order to use the extracorporeal circuit support according to the invention for patient transports between the operating room and the MRT room as well, but also for use with ECMO patients, it may be advantageous if the compressed air used as the working medium can be generated by means of a preferably portable or transportable compressor of the extracorporeal circuit support.

A further developed embodiment according to the invention in which the gas expansion motor is configured as a ferrite-free gas expansion motor, and in particular as a ferrite-free radial piston motor, is preferred.

According to one advantageous embodiment, a control unit for controlling a pump unit may be provided, the pump unit comprising at least the gas expansion motor and the liquid primary pump. In this case, the control unit may be arranged at a distance from the pump unit. In this case, the pump unit may be operable at a distance from the control unit. Thus, the pump unit may be operated close to the patient and therefore in closer proximity to an MRT than the control unit. Effects of the MRT on the control unit, and vice versa, may thus be reduced or avoided. The requirements placed on the control unit in relation to MR safety may also be reduced.

The control unit may be referred to as a controlling unit.

In this case, the control unit may be connected to the pump unit via a compressed-air line and/or an electrical connection. It is advantageous for the electrical connection to be electromagnetically shielded.

In one advantageous configuration, a flow sensor which is adapted to measure a flow rate inside a liquid line of the extracorporeal circuit support may be provided. The flow rate of the blood may thus be accurately regulated. It is particularly advantageous for this to be a non-invasive flow sensor. Such a sensor may on the one hand be used repeatedly, and on the other hand a detrimental effect of the flow sensor on the blood is avoided.

It is advantageous to provide a rotational-speed sensor which is adapted for at least indirect determination of a rotational speed of the liquid primary pump. This may for example be a rotational speed of a driven shaft located between the transmission and the liquid primary pump. Preferably, the rotational-speed sensor may be electromagnetically shielded and, for example, arranged in a copper shielding. An electrical connection, preferably a signal connection, of the rotational-speed sensor to a control unit is preferably likewise configured to be electromagnetically shielded. Interference with the control unit by parasitic signals fed into an electrical line may thus be avoided.

The rotational-speed sensor may also be configured as an encoder. It is therefore possible to record the rotational speed of a drive shaft or driven shaft and to use this for accurate driving of the gas expansion motor or of the transmission. If both a flow sensor and a rotational-speed sensor are provided, the flow rate and rotational speed may function interdependently as control values. Deviations from a known ratio of the two values with respect to one another may indicate possible interferences, for example due to the operation of the MRT.

According to one advantageous embodiment, a marking which allows deduction of an alignment of a drive shaft associated with the gas expansion motor is provided on a housing of the extracorporeal circuit support.

This may be a visually perceptible marking, for instance an arrow. The alignment of the drive shaft is externally visible and the circuit support may thus be aligned in an external magnetic field so that an interfering effect of the magnetic field, for example in the form of braking and/or blocking of the drive, of the gas expansion motor and/or of the drive shaft, is reduced and/or avoided.

Furthermore, in order to achieve the object mentioned in the introduction, according to the invention, in an MRT arrangement having a magnetic resonance tomograph (MRT) and an extracorporeal circuit support according to the invention, a drive shaft associated with the gas expansion motor is aligned parallel to a longitudinal midaxis of an annular arrangement of coils. Alternatively or additionally, this drive shaft may be aligned parallel to a longitudinal midaxis of an examination opening of the MRT.

An interfering effect of the MRT on the drive of the circuit support may therefore be reduced or avoided. In a different alignment of the drive shaft or of the gas expansion motor, an interfering effect of the MRT may be manifested in the form of braking of the drive. The examination opening is generally designed cylindrically and is defined by the opening of the annular arrangement of coils. The coils comprise, for example, coils for generating the magnetic field and radiofrequency coils. The adjustment of the correct alignment of the drive shaft may, in particular, be assisted by the above-described marking on a housing of the circuit support.

### BRIEF DESCRIPTION OF THE DRAWINGS

Developments according to the invention may be found from the following description of an exemplary embodiment according to the invention in conjunction with the claims and the drawing. The invention will be described in more detail below with the aid of a preferred exemplary embodiment.

FIG. 1 represents a schematic drawing of an extracorporeal circuit support which may be used in the immediate vicinity of an MRT and therefore of a patient.

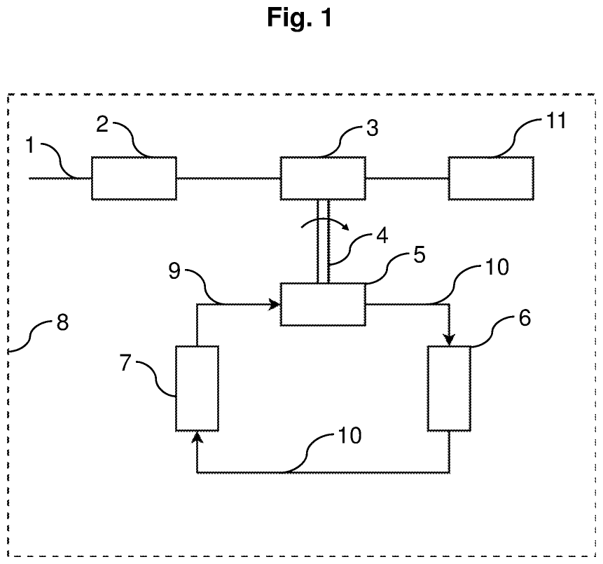

FIG. 2 shows a further schematized representation of the extracorporeal circuit support with further components.

### DETAILED DESCRIPTION

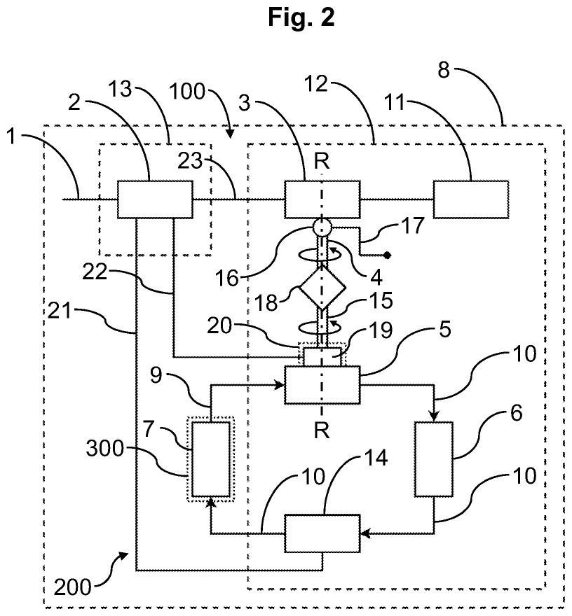

The extracorporeal circuit support is configured as an apparatus for the extracorporeal circuit support of a patient 7, and is denoted as a whole in FIG. 2 by 100. FIG. 2 furthermore shows an MRT arrangement 200 according to the invention, which comprises an extracorporeal circuit support 100 and an MRT 300.

In the exemplary embodiment shown here, the extracorporeal circuit is used in an MRT room 8. The extracorporeal circuit support 100 shown here has a blood or liquid primary pump 5, the pump inlet of which can be connected via at least one first liquid line 9 and the pump outlet of which can be connected via at least one second liquid line 10 to the circulatory system of a patient 7. The extracorporeal circuit support 100 shown here has an oxygenator 6 which is intended to enrich with oxygen the blood conveyed in the at least one second liquid line 10 to the patient 7 and to eliminate the carbon dioxide contained in the blood. In order to make the pump drive assigned to the liquid primary pump 5 conditionally MR-compatible as well, besides the liquid primary pump 5 and the oxygenator 6, this pump drive is configured here as a gas expansion motor 3. This gas expansion motor 3 has a drive shaft 4, which is operatively connected to the blood or liquid primary pump 5. This drive shaft 4 and all further shafts located in the drive train between the pump drive and the liquid primary pump 5 are produced from preferably stiff fiber-reinforced composites, for example carbon or glass fiber-reinforced composites, and mounted in ceramic ball bearings and/or plastic bushes. The compressed gas used as the working medium is supplied to the gas expansion motor 3 via a supply line 1. A regulating valve 2, by which the volume flow rate of the compressed gas supplied to the gas expansion motor 3 as the working medium can be regulated, is interconnected in the supply line 1. This regulating valve 2 is preferably designed as a piezo valve.

In order to be able to regulate the rotational speed of the shaft 4 driven by the gas expansion motor 3 in a wide rotational-speed range and to be able to vary the torque of the gas expansion motor 3, a transmission may be provided in the drive train between the gas expansion motor 3 and the liquid primary pump 5. The transmission (not further represented here) may be designed as a continuously variable transmission (CVT), for example as a v-belt variator or as a NuVinci transmission. It is also possible for the transmission to be configured as a gear transmission or planetary transmission, or as a hydrodynamic torque converter.

The liquid primary pump 5 may be configured as a dynamic pump, for example as a rotary pump or as an axial-flow pump. It is also possible for the liquid primary pump 5 to be configured as an impeller pump. In the exemplary embodiment shown here, the liquid primary pump 5 is configured as a roller pump or peristaltic pump. The conveyed medium is in this case contained in a tube, over which one or more rollers roll. The heart-lung machine, consisting essentially of the liquid primary pump 5 configured as a pump head, the gas expansion motor 3 used as the pump drive, the oxygenator 6 and the cardiopulmonary bypass formed from the liquid lines 9 and 10, has a control connection for parameter adjustment and parameter monitoring to a control unit 13, which may for example be configured as a preferably portable data-processing system or as a notebook. By virtue of the design according to the invention of the essential components of the heart-lung machine schematically represented here, the latter may be placed inside the MRT environment in an MRT room. In the exemplary embodiment shown, the control unit comprises the regulating valve 2, although it may also be configured separately.

The primary liquid pump 5 driven in rotation with the aid of the gas expansion motor 3 is configured here as a peristaltic pump, which has a plurality of rollers that roll in the pump head on the tube carrying the conveyed medium. The squeezing of the tube and the correction of the occlusion setting are of fundamental importance in order to avoid a backflow with an increase in the kinetic energy (non-occlusive or sub-occlusive), a reduction of the lifetime of the tubes due to spallation (over-occlusion) or hemolysis. A “just-occlusive” setting without retrograde flow and with minimized spallation has in this case been found to be an optimal setting. The blood of the patient 6 is supplied to the blood or primary liquid pump 5 via a first liquid line 9, the blood or primary liquid pump 5 subsequently conveying the blood via a second liquid line 10 into which the oxygenator 6 and optionally also a heat exchanger is interconnected. In order to measure the blood circulation rate of the patient accurately, an ultrasonic flow rate probe may be used, while the pressure is measured with the aid of a pressure transducer and forwarded to the control unit. The oxygenator 6 used, the cannulas, the tubes used as liquid lines, and the blood pump are conditionally MR-compatible or MR-safe and may be placed as close as possible to the patient in order to minimize the filling volume of the extracorporeal circuit.

The required compressed gas, here compressed air, is supplied to the gas expansion motor 3 via the supply line 1. The regulating valve 2 interconnected into the supply line 1 regulates the air flow which drives the gas expansion motor 3. The air flowing out from the gas expansion motor 3 is guided away from the patient and diffused through a sound absorber 11. Although the gas expansion motor 3 and the sound absorber 11 can be placed next to the MRT, the pressure regulating valve 2, configured here as a piezo valve, is located outside the 20 mT line, while conversely the gas expansion motor 3, the noise absorber 11 assigned to the gas expansion motor 3, the drive shaft 4, the liquid primary pump 5 and the oxygenator 6 together with the patient 7 are located inside the 20 mT line of the MRT room 8. The control unit, formed for example by a notebook, and the associated displays may be located in a shielded housing which prevents the emission of radiofrequency electromagnetic radiation into the MRT environment.

The various elements of the extracorporeal circuit support 100 shown may be categorized into a pump unit 12 and a control unit 13. The control unit 13 is configured at a distance from the pump unit 12 and is connected to the latter via a fluid line, here a compressed-air line 23, and electromagnetically shielded electrical connections 21, 22. The control unit 13 furthermore comprises a non-invasive flow sensor 14, by which the flow rate of the blood is determined.

A connector 16 for a removable emergency crank 17 is provided on the drive train of the extracorporeal circuit support 100, or more precisely on the drive shaft 4 between the gas expansion motor 3 and the transmission 18. The moving parts of the transmission 18 and of the liquid primary pump 5 are designed to be metal-free and MR-safe, and they comprise parts made of plastic and ceramic.

The connector 16, and therefore also the emergency crank 17, are in this case arranged in the drive train before the transmission 18. Thus, a drive movement applied manually via the emergency crank 17 may be applied via the transmission 18 and converted by the latter. By this transmission support, it is more readily possible to achieve a particular minimum rotational speed of the liquid primary pump 5, which is necessary for proper operation of the liquid primary pump 5, during manual emergency operation as well.

A rotational-speed sensor 19 is furthermore configured, which determines a rotational speed of a driven shaft 15 located between the transmission 18 and the liquid primary pump 5. The rotational-speed sensor 19 is configured as an encoder and is enclosed by an electromagnetic shielding 20.

The patient 7 is enclosed by an MRT 300. The extracorporeal circuit support 100 and the MRT 300 form an MRT arrangement 200 according to the invention, in which a rotation axis R of the drive shaft 4 of the gas expansion motor 3 is aligned parallel to a longitudinal midaxis of an annular arrangement of coils and an examination opening of the MRT 300.

MR-safe objects are defined as objects which entirely consist of electrically non-conductive, non-metallic and non-magnetic materials, and therefore represent no danger and furthermore generate no image artifacts, while objects which are designated as conditionally MR-compatible are allowed in MRT environments under defined conditions and do not cause image interference. MR-unsafe objects represent an unacceptable risk for the patient and are therefore prohibited. Most MRT rooms have a line marked on the floor, which indicates the 20 mT line. Conditionally MR-compatible electronic devices, for example MR anesthesia monitors or MR medical ventilators, should remain outside this 20 mT line. According to the international MRT safety standard, a conditionally MR-compatible device must create no artifacts during the imaging. The pump unit, comprising the gas expansion motor 3 and the liquid primary pump 5, of the extracorporeal circuit support 100 represented here is produced entirely from MR-safe and conditionally MR-compatible materials. No tests on displacement forces or magnetic field-induced torque are therefore necessary, and the testing of image artifacts may also be omitted since none of the devices is placed directly in or directly next to the image region of the MRT. Apart from the control unit (not further represented here), essentially all components represented here may be installed at a distance of about 1 m from the isocenter of the MRT. Only the piezo valve 2 used as the regulating valve and a special Faraday cage with control and data recording are to be placed about 3 m away from the isocenter of the MRT. These distance indications are recommended in connection with a 3-tesla MRT, and should also apply for a 1-tesla and 1.5-tesla MRT.

### LIST OF REFERENCE SIGNS

1 compressed-gas or compressed-air supply line
2 regulating valve, in particular piezo valve
3 gas expansion motor
4 drive shaft
5 blood or liquid primary pump
6 oxygenator
7 patient
8 MRT room
9 first liquid line
10 second liquid line
11 noise or sound absorber
12 pump unit
13 control unit
14 flow sensor
15 driven shaft
16 connector
17 emergency crank
18 transmission
19 rotational-speed sensor
20 shielding
21 connection
22 connection
23 compressed-air line
100 extracorporeal circuit support
200 MRT arrangement
300 MRT
R rotation axis

1. An extracorporeal circuit support (100), comprising:
a liquid primary pump (5) having a pump inlet of which is adapted for connection via at least one first liquid line (9) and a pump outlet of which is adapted for connection via at least one second liquid line (10) to a circulatory system of a patient (7);
an oxygenator (6) for enriching with oxygen blood conveyed in the at least one second liquid line (10); and
a pump drive which drives the liquid primary pump (5), the pump drive is MR-compatible and is configured as a gas expansion motor (3).

2. The extracorporeal circuit support (100) as claimed in claim 1, further comprising at least one piezo valve (2) by which a volume flow rate of compressed gas supplied to the gas expansion motor (3) as a working medium is regulatable.

3. The extracorporeal circuit support (100) as claimed in claim 1, wherein the gas expansion motor (3) has a drive shaft (4) which is operatively connected to the liquid primary pump (5).

4. The extracorporeal circuit support (100) as claimed in claim 1, further comprising at least one connector (16) for an emergency crank (17) provided on a drive train for the liquid primary pump.

5. The extracorporeal circuit support (100) as claimed in claim 1, further comprising a transmission (18) in a drive train between the gas expansion motor (3) and the liquid primary pump (5).

6. The extracorporeal circuit support (100) as claimed in claim 5, wherein the transmission (18) comprises a continuously variable transmission (18), a gear transmission or planetary transmission, or a hydrodynamic torque converter.

7. The extracorporeal circuit support (100) as claimed in claim 1, wherein a drive shaft (4) of the gas expansion motor (3) is formed from a fiber-reinforced composite.

8. The extracorporeal circuit support (100) as claimed in claim 1, wherein a drive shaft (4) of the gas expansion motor (3) is mounted with at least one of ceramic ball bearings or plastic bushes.

9. The extracorporeal circuit support (100) as claimed in claim 1, wherein the liquid primary pump (5) comprises a roller pump, a peristaltic pump or a flexible-tube pump.

10. The extracorporeal circuit support (100) as claimed in claim 1, wherein the liquid primary pump (5) is a dynamic pump.

11. The extracorporeal circuit support (100) as claimed in claim 1, wherein the liquid primary pump (5) comprises a rotary pump or an axial-flow pump.

12. The extracorporeal circuit support (100) as claimed in claim 1, wherein the liquid primary pump (5) comprises an impeller pump.

13. The extracorporeal circuit support (100) as claimed in claim 5, wherein moving parts of at least one of the transmission (18) or of the liquid primary pump (5) are at least one of metal-free, consist of plastic or consist of ceramic.

14. The extracorporeal circuit support (100) as claimed in claim 1, wherein a working medium for the gas expansion motor (3) comprises compressed air or nitrogen.

15. The extracorporeal circuit support (100) as claimed in claim 14, further comprising a compressor that generates the compressed air used as the working medium.

16. The extracorporeal circuit support (100) as claimed in claim 1, wherein the gas expansion motor (3) comprises a multi-disk, turbine, gear, axial piston motor, or radial piston motor.

17. The extracorporeal circuit support (100) as claimed in claim 1, wherein the gas expansion motor (3) is a ferrite-free gas expansion motor (3).

18. The extracorporeal circuit support (100) as claimed in claim 1, further comprising a control unit (13) for controlling a pump unit (12) comprising at least the gas expansion motor (3) and the liquid primary pump (5), and the control unit (13) is arranged at a distance from the pump unit (12).

19. The extracorporeal circuit support (100) as claimed in claim 18, wherein the control unit (13) is connected to the pump unit (12) via at least one of a compressed-air line (23) or an electromagnetically shielded electrical connection (21, 22).

20. The extracorporeal circuit support (100) as claimed in claim 1, further comprising a non-invasive flow sensor (14) for measuring a flow rate inside the at least one first or the at least one second liquid line (9, 10).

21. The extracorporeal circuit support (100) as claimed in claim 1, further comprising a rotational-speed sensor (19) which is adapted for at least indirect determination of a rotational speed of the liquid primary pump.

22. The extracorporeal circuit support (100) as claimed in claim 1, further comprising a marking which allows deduction of an alignment of a drive shaft (4) of the gas expansion motor (3).

23. The extracorporeal circuit support (100) as claimed in claim 1, wherein the extracorporeal circuit support (100) is configured as a heart-lung machine or as extracorporeal membrane oxygenation (ECMO).

24. An MRT arrangement (200) having a magnetic resonance tomograph (MRT) (300) and the extracorporeal circuit support (100) as claimed in claim 1, wherein a rotation axis (R) of a drive shaft (4) of the gas expansion motor (3) is aligned parallel to a longitudinal mid-axis of at least one of an annular arrangement of coils or an examination opening of the MRT (300).
